(19) United States
(12) Patent Application Publication
(10) Pub. No.: US 2026/0308701 A1
(43) Pub. Date: Oct. 8, 2026

(54) MICROFLUIDIC DEVICE FOR IN-FIELD PURIFICATION, AMPLIFICATION, AND DETECTION OF DNA FROM MYCOTOXIN-PRODUCING FUNGI

(71) Applicant: PURDUE RESEARCH FOUNDATION, West Lafayette, IN (US)

(72) Inventors: Joseph Paul Robinson, West Lafayette, IN (US); Bartlomiej P. Rajwa, West Lafayette, IN (US); Euiwon Bae, West Lafayette, IN (US); Cole Reynolds, West Lafayette, IN (US); Xi Wu, West Lafayette, IN (US)

(21) Appl. No.: 19/479,013

(22) PCT Filed: Apr. 30, 2024
(86) PCT No.: PCT/US2024/027025

§ 371 Date: Oct. 27, 2025

Related U.S. Application Data
(60) Provisional application No. 63/499,236, filed on Apr. 30, 2023.

(51) Int. Cl. C12Q 1/6895; B01L 3/00; B01L 7/00

(52) U.S. Cl. CPC C12Q 1/6895; B01L 3/502715; B01L 7/52; B01L 2200/027; B01L 2200/0647; B01L 2200/18; B01L 2300/12; B01L 2300/165; B01L 2300/1805; C12Q 2600/156

(57) ABSTRACT

Methods of testing grain samples for the presence of mycotoxin-producing fungi using portable devices are disclosed. The design and fabrication of such devices to practice the disclosed methods allow for rapid and portable in-field detection of mycotoxin-producing fungi prior to harvest and potential contamination of the food supply with infected grain.

### CROSS REFERENCE TO RELATED APPLICATIONS

This application is related to and claims priority to U.S. Provisional Patent Application Ser. No. 63/499,236, filed on Apr. 30, 2023

### GOVERNMENT FUNDING

This invention was made with government support under Agreement Nos. 59-8072-1-002 and 58-8042-0-061 awarded by the Department of Agriculture, Agricultural Research Service. The government has certain rights in the invention.

### FIELD

The claimed technology relates generally to the field of analytical chemistry, and, more particularly, to rapid and portable in-field detection of mycotoxin-producing fungi.

### BACKGROUND

Certain species of fungi can infect and contaminate crops with mycotoxins. Infection of crops by these fungal species can cause mycotoxins to enter food streams through products made from crops or through animal feed. Mycotoxins can cause a variety of negative health outcomes. Food regulatory agencies around the world such as the U.S. Food and Drug Administration (FDA) have set maximum allowable concentrations for various mycotoxins in food. The concentration of such mycotoxins may increase drastically throughout the processing steps from raw plants to processed food products. Because of this, mycotoxins are typically tested for after harvesting and processing. Testing after harvesting and processing has inherent inefficiencies because growers cannot apply fungicides effectively, limit cross-contamination, or avoid wasting resources harvesting infected or preventatively treating uninfected crops.

Fusarium graminearum is a fungal species that infects cereal crops and produces dangerous mycotoxins. The plant disease is called Fusarium head blight (FHB) or scab. FHB can persist in fields over multiple years as Fusarium graminearum can overwinter on dead plants. The estimated value of yield forgone due to FHB of wheat and barley in the United States is estimated to be $1.469 billion annually. Deoxynivalenol (DON) is a mycotoxin produced by Fusarium graminearum and can produce symptoms such as vomiting, nausea, headaches, and fever when consumed. The FDA has set a maximum concentration of 1 ppm of DON in human and 5 ppm in animal feed. Due to predicted climate changes, FHB is expected to become more prevalent in coming years. There exists a need for devices and methods for testing crops for the presence of mycotoxin-producing fungi prior to the harvest and processing of such crops.

### SUMMARY

The consumption of mycotoxins generated by fungi can have severe effects on the health of both humans and animals. These toxins can exist at dangerous levels in food products made from crops that have been infected with mycotoxin-producing fungi. Numerous methods have been developed for detecting mycotoxins in order to divert contaminated commodities from the food supply, but only allow for reactive, not preventive approaches. Furthermore, under favorable conditions toxin-producing fungi can continue to produce mycotoxins during storage and throughout the crop processing stages. By identifying mycotoxin-producing fungal species on crops or commodities, remediation such as fungicide application can be carried out, preventing the spread of infection and potential contamination of healthy crops, reducing waste of resources and ultimately improving food safety. Loop-mediated isothermal amplification (LAMP) has advantages for portable DNA detection due to its isothermal nature, resistance to matrix inhibitors, and the possibility of a long shelf-life when reagents are dried onto a matrix. The developed microfluidic device allows for the homogenized wheat sample input after DNA extraction. The microfluidic device functions as a disposable cassette and can be heated by an independent, portable, isothermal heating device. The LAMP assay may be combined with calcein for fluorescence detection. In this experiment, Fusarium graminearum, a trichothecene mycotoxin producer, was used as a proof-of-concept for the device with a LAMP assay targeting the gaoA gene, which codes for the enzyme galactose oxidase (GO), a unique enzyme produced by only a few other fungal species. The presence of Fusarium graminearum was detected in contaminated wheat samples utilizing the described methods, indicating the potential detection of mycotoxin-producing fungi.

In one aspect, methods for identifying mycotoxin-producing fungal species on crops in the field and devices for practicing such methods. One example method includes the steps of procuring a grain sample, optionally directly in the field, extracting DNA from the grain sample, isothermally amplifying the extracted DNA, identifying any DNA from mycotoxin producing fungus as amplified target DNA, and fluorescing the products of amplified target DNA to yield quantitative information relating to the presence of DNA from mycotoxin-producing fungus, where the amplifying, identifying, and fluorescing steps are performed in the same microfluidic chamber platform. The extracting step may comprise milling the grain sample and then lysing the milled grain sample. The extracting comprises solid phase extraction using one of silica membranes, glass particles, diatomaceous earth, magnetic beads, anion exchange material, and cellulose matrix. Optionally, the sample may be washed with a chaotropic agent between the extracting and amplifying steps. In another example, the amplifying and identifying includes a paper-based nucleic acid amplification test. The amplifying may be a loop-mediated isothermal amplification, and such loop-mediated isothermal amplification may use four to six or more primers. In another example, the DNA from more than one species or variety of mycotoxin-producing fungus may be extracted, amplified, identified, and fluoresced by the disclosed methods, either sequentially or simultaneously.

In another aspect, a method of in-field detection of mycotoxin producing fungus, including the steps of collecting potentially infected plant tissue samples, grinding potentially infected plant tissues to yield ground tissue material, lysing ground tissue material to extract DNA, purifying extracted DNA to yield purified DNA, performing a fluorescence-based loop-mediated isothermal amplification assay on the purified DNA, and measuring the amount of fluorescence from DNA from mycotoxin-producing fungus to yield a quantitative measurement. The grinding and lysing steps may be performed in a first device and the purifying, amplification, and fluorescence steps performed in a second device. The first device may be a portable bead mill and the second device a portable microfluidic device or platform. The portable microfluidic device may include a housing, a microfluidic chamber for purifying extracted DNA and performing a fluorescence-based loop-mediated isothermal amplification assay on the purified DNA positioned within the housing and operationally connected to the bead-bashing chamber, and a power source positioned within the housing operationally connected to the microfluidic chamber. In other examples, the steps are performed in a single device.

In still another aspect, a portable microfluidic device for extracting fungal DNA from grain samples and performing qualitative and quantitative analysis of the same includes a housing, a bead-bashing chamber enclosed within the housing for extracting DNA from plant tissue samples, a microfluidic chamber for purifying extracted DNA and performing a fluorescence-based loop-mediated isothermal amplification assay on the purified DNA positioned within the housing and operationally connected to the bead-bashing chamber, and a power source positioned within the housing and operationally connected to the microfluidic chamber. Optionally, the power source is operationally connected to the bead-bashing chamber. Optionally, the power source is operationally connected to a control device having a processor, memory, optional wireless or wired information transmission capabilities, and is operationally connected to and can control the components of the portable microfluidic device. The microfluidic chamber may further include an input port, at least two fluid distribution channels fluidically connected to the input port, and at least one outlet port fluidically connected to each of the at least two fluid distribution channels. Fluid flow through the at least two fluid distribution channels may be controlled by selectively covering the outlet ports. The outlet ports may be selectively covered by hydrophobic microfluidic tape. The device may further include at least one LAMP reaction chamber fluidically connected to one of the fluid distribution channels. The device may further include a heating device configured and arranged to warm the microfluidic chamber to a desired temperature, for example, to at least 65 degrees C. The microfluidic chamber may be made from PMMA. The input port may be a luer lock style port.

### BRIEF DESCRIPTION OF THE DRAWINGS

FIG. 1 is an example of the methods of testing for mycotoxin-producing fungal species according to one embodiment.

FIG. 2 is a cross sectional view of one example of a device used to test for mycotoxin-producing fungal species according to one embodiment.

FIG. 3 is a graph depicting the relationship between DNA concentration and

relative fluorescence according to one embodiment.

FIG. 4 is a graph depicting results of a LAMP test matrix and a negative amplification control according to one embodiment.

FIG. 5 is a partial cross sectional view of one example method of fabricating a microfluidic device according to one embodiment.

FIG. 6 is a schematic view of an example of a microfluidic device according to one embodiment.

FIG. 7 is a graph depicting time require for a heating device to raise the temperature inside a LAMP chamber according to one embodiment.

FIG. 8 is an image of gel electrophoresis showing detectable products in a LAMP reaction at various temperatures.

FIG. 9 is an image of gel electrophoresis showing detectable products in a LAMP reaction over time.

FIG. 10 is a schematic view of an example of a microfluidic device according to another embodiment.

FIG. 11 is a schematic view of an example of a microfluidic device according to still another embodiment.

FIG. 12 is one example of the fluorescence detection mechanism used by the methods disclosed herein.

### DESCRIPTION

For the purposes of promoting an understanding of the principles of the claimed technology and presenting its currently understood best mode of operation, reference will now be made to the embodiments illustrated in the drawings and specific language will be used to describe the same. It will nevertheless be understood that no limitation of the scope of the claimed technology is thereby intended, with such alterations and further modifications in the illustrated device and such further applications of the principles of the claimed technology as illustrated therein being contemplated as would normally occur to one skilled in the art to which the claimed technology relates.

Before the present methods, implementations, and systems are disclosed and described, it is to be understood that this invention is not limited to specific synthetic methods, specific components, implementation, or to particular compositions, and as such may, of course, vary. It is also to be understood that the terminology used herein is for the purpose of describing particular implementations only and is not intended to be limiting. As used in the specification and the claims, the singular forms “a,” “an” and “the” include plural referents unless the context clearly dictates otherwise. Ranges may be expressed in ways including from “about” one particular value, and/or to “about” another particular value. When such a range is expressed, another implementation may include from the one particular value and/or to the other particular value. Similarly, when values are expressed as approximations, for example by use of the antecedent “about,” it will be understood that the particular value forms another implementation. It will be further understood that the endpoints of each of the ranges are significant both in relation to the other endpoint, and independently of the other endpoint. “Optional” or “optionally” means that the subsequently described event or circumstance may or may not occur, and that the description includes instances where said event or circumstance occurs and instances where it does not. Similarly, “typical” or “typically” means that the subsequently described event or circumstance often though may not occur, and that the description includes instances where said event or circumstance occurs and instances where it does not.

The need exists for a method of detecting the presence of Fusarium graminearum in the field or crops as a proxy for risk of toxins such as DON contamination. For the method to be applicable to the need, it is desirable to be portable, inexpensive, and easy to use. To meet these requirements, a loop-mediated isothermal amplification (LAMP) assay is one possible detection modality. LAMP in one example uses DNA samples that can be purified using silica membrane binding. LAMP is similar to polymerase chain reaction (PCR) in that it amplifies target DNA sequences. LAMP provides some advantages over PCR as a portable detection method because it is isothermal and does not require a bulky heat source, can be used on less pure DNA samples, and the reagents can be dried to a matrix to extend the shelf life without a freezer.

A LAMP assay produces a fluorescent readout using calcein dye. When pyrophosphates are generated by LAMP amplification, manganese ions in solution disassociate from the calcein molecules. Magnesium ions in solution bind to the calcein ions, which enter the calcein into a fluorescent state. Calcein fluorescence can then be measured through exciting with blue light and detecting green light.

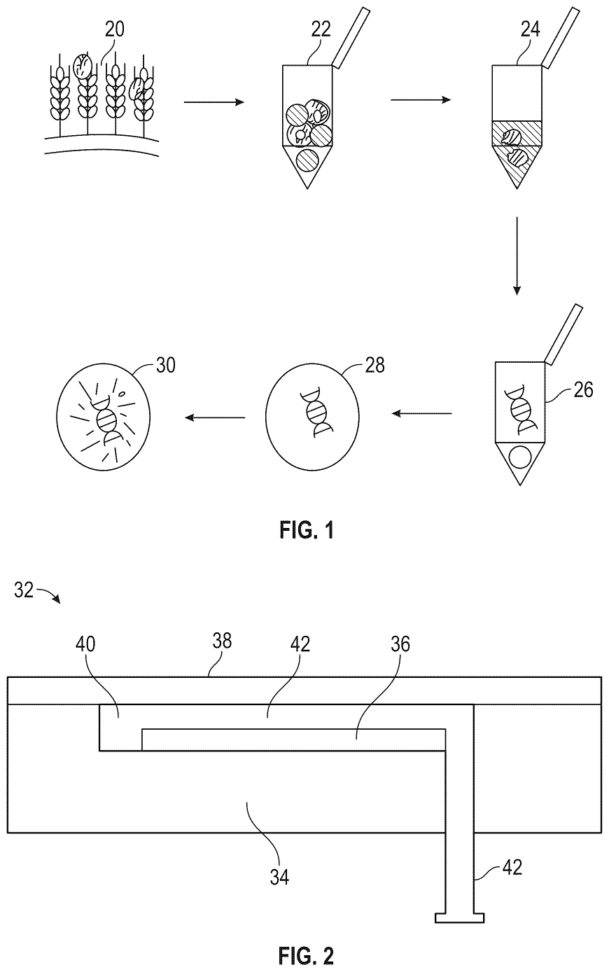

As seen in FIG. 1, one example of an overall detection process begins with the collection of potentially infected wheat 20 florets or grain. Tissues may be first visually inspected for physical signs of fungal infection. Typical FHB symptoms include premature bleaching of florets and/or pink fungal hyphae or spots on grain. After grinding potentially infected tissues inside of a tube 22, an aqueous solution and beads are added to the tube. The tube is placed inside of a portable bead basher/miller 24, which lyses the fungal cells. The lysing of the fungal cells releases the DNA from the membrane into the solution. A chaotropic agent is then added to the solution so that when the solution flows through a silica membrane, the DNA binds to the silica membrane 26. The silica membrane is then washed of unwanted molecules such as proteins using a chaotropic agent and chelating agent solution. Afterwards, the DNA is eluted using a salt solution giving a purified DNA solution. The DNA solution is then added to a microfluidic chip containing the LAMP reagents 28. A LAMP assay with 6 primers targeting the gaoA gene, which has a relatively high specificity to Fusarium graminearum, is then dried onto polypropylene matrices in the microfluidic device. A portable heating device is used to heat to the isothermal LAMP reaction. A portable fluorescence detection device is used to detect the fluorescence intensity to determine the presence of the target fungal DNA 30.

Examples of suitable microfluidic devices typically will be designed to be inexpensive, easy to use, portable, and disposable. Because the intended users of the device are people who do not have laboratory experience, the device is typically easy to use. In some examples the in-field detection device is portable and does not require bulky equipment. LAMP reagents in solution typically require storage in a freezer, but can be dried to a matrix to extend their shelf life at room temperature. Examples of microfluidic devices use channels to appropriately distribute the DNA solution to the incorporated LAMP reagent-soaked matrices.

Example devices may be manufactured by engraving a channel design into polymethyl methacrylate (PMMA). The LAMP reagent-soaked matrices are placed inside of the design. Hydrophobic microfluidic tape is then placed across the channels to close the top for fluid flow. For ease of use, the fluid flow may be controlled by the pressure generated by a syringe. The device can then be placed directly inside of a heating or fluorescence detection device.

FIG. 2 shows a partial cross sectional view of an example of a micro-fluidic device 32 for practicing the methods disclosed herein according to one embodiment. In this particular example, the micro-fluidic device 32 PMMA is engraved with a pattern 34, LAMP reagent-soaked matrices 36 are placed inside the pattern 34, and microfluidic tape 38 is placed over the pattern 34 to create microfluidic channels 40. DNA solution 42 is injected through from a syringe through a luer lock or other suitable connector 42. In one example, to be applicable to assessing trichothecene mycotoxin risk, a device of the correct sensitivity to Fusarium graminearum DNA is fabricated having approximately 6.25 μg/mL of fungal DNA as the provided limit of detection (LOD).

In one example a serial dilution of Fusarium graminearum was created for LOD determination. The serial dilution of known LAMP reagents were added to polypropylene filter matrices. An additional matrix was included with Fusarium graminearum DNA at the highest concentration, but no LAMP primers as a negative control. A positive control with calcein was added as well. The matrices were inserted into chambers identical to those of the microfluidic device. The LOD was calculated where Sy is the standard deviation of the negative control and S is the slope of the response in the equation:

$$LOD = 3.3 ( \frac{{S}_{y}}{S} )$$

The fluorescence was measured using a well plate reading device (e.g., a Synergy HT® well plate reader by Biotek, Vermont, USA). Two measurements were taken in two different locations of each serial dilution and averaged. The relative fluorescence was calculated by subtracting the negative control fluorescence and dividing by the positive control fluorescence to normalize day to day variability.

In another example, samples of ground wheat infected with Fusarium graminearum were collected. DNA was extracted from these samples using the process described above. The DNA was then injected into a device containing a LAMP reagent matrix, a negative amplification control matrix with all LAMP reagents except for primers, a calcein and magnesium positive control matrix, and a calcein and manganese negative fluorescence control matrix. To successfully detect the Fusarium graminearum DNA, the fluorescence of the test matrix including the primers should be statistically different than the matrix containing no primers.

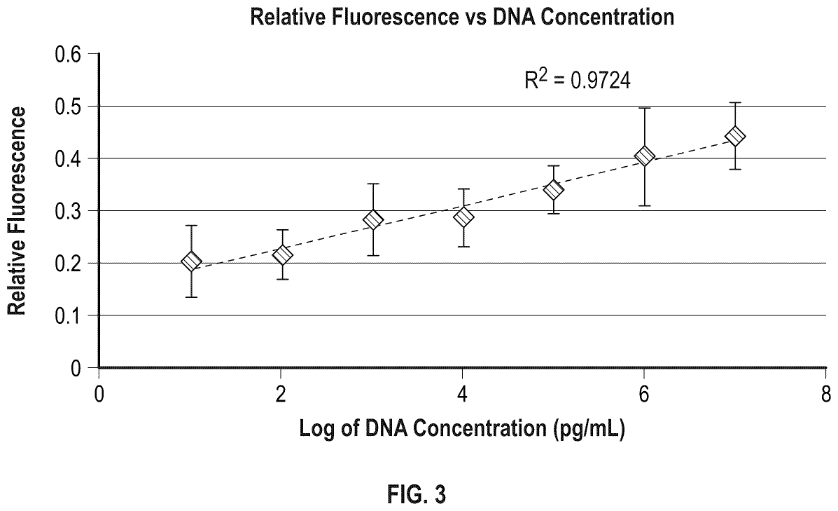

The fluorescence was measured using a well plate reader. Two measurements were taken in two different locations on each matrix and averaged. The relative fluorescence was calculated by subtracting the negative control fluorescence and dividing by the positive control fluorescence to normalize day to day variability. The results of the LOD experiment are shown in FIG. 3 with relative fluorescence appearing to have a linear relationship with the log of DNA concentration. Five serial dilutions were tested. The LOD was calculated using the equation for LOD above to be 0.408 μg/mL, which is less than the 6.25 μg/mL required LOD.

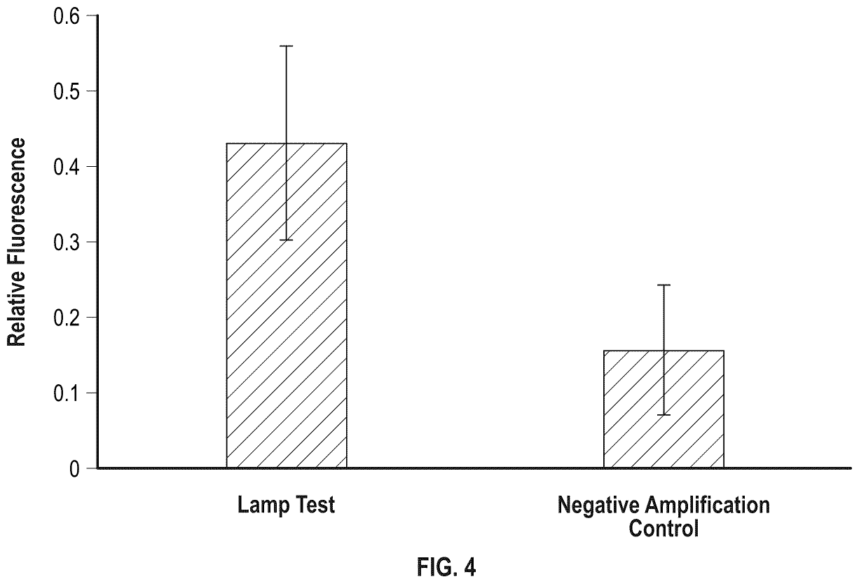

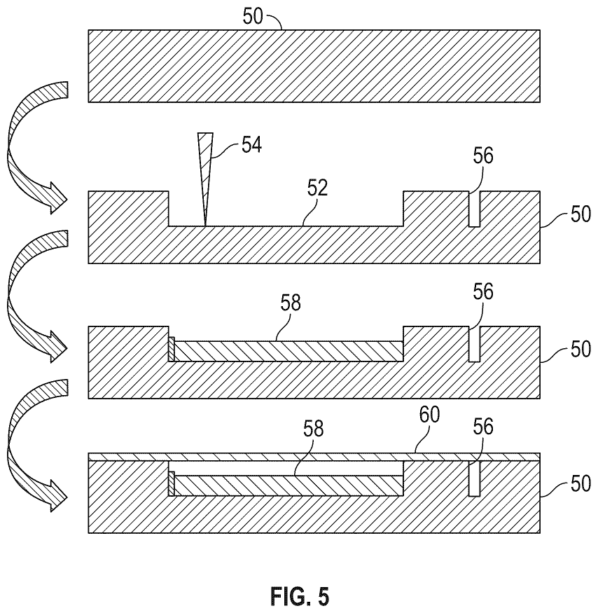

The results of one microfluidic experiment are shown in FIG. 4 with the LAMP test matrix having a higher average relative fluorescence than the negative amplification control (n=5). A two-sample t-test showed a statistical difference between the average relative fluorescence of the two groups (p<0.05). It was concluded that the LAMP reaction did produce fluorescence in the presence of the target DNA sequence.

The purpose of this example was to show proof-of-concept in the detection of fungal DNA in a portable format. The limit of detection experiment resulted in a lower LOD than the required LOD. Higher fluorescence was detected in the LAMP test matrix than in the negative controls, indicating that the fungal DNA was successfully detected. The format of the device is such that more chambers for matrices can be added. With each chamber containing LAMP assays with unique target primer sequences, LAMP assays could be multiplexed. With the multiplexing of multiple target DNA sequences, multiple kinds of infectious fungal species can be detected. The portable microfluidic device presented has the capacity to be a portable detection device for many mycotoxin-producing fungal species.

To test crop samples, the part of the plant that is used in food production is homogenized. Because the fungus grows on the inside of the plant, homogenizing the samples allows for chemical to react with the fungus that can growing deeper within the plant and not just on the outer surface. The homogenization process usually involves bashing the sample with beads or other suitable means to create a powder. That powder is then put through suitable extraction and filtration steps depending upon the analysis method. Lysis of fungal cells is used in one example to break apart the cell membrane to expose the DNA. Common methods of fungal cells include surfactants, freezing and thawing, sonication, bead milling or bashing, and grinding. Bead milling has been shown to have a high yield compared to other methodologies. In addition to being a high yielding technique, it also is considerably more portable than the other methodologies. The only equipment required for bead milling is a homogenizer that can rapidly oscillate a tube in a linear motion. These homogenizers are available in handheld, battery-operated formats, making them ideal for in-field lysis.

The two most common approaches to DNA extraction are chemically driven methods and solid phase extraction methods. Chemically driven methods generally require a centrifuge and other large laboratory equipment to perform. Due to these constraints, DNA extraction for in field testing is more suited for solid phase extraction methods. Solid phase extraction methods use a solid material that can bind to DNA in certain pH or salt concentration conditions. The DNA can then be eluted from the material by again changing the pH or salt concentration conditions. Examples of materials that are used for solid phase extraction include silica membranes, glass particles, diatomaceous earth, magnetic beads, anion exchange material, and cellulose matrix. Glass particles, diatomaceous earth, magnetic beads, and anion exchange material are costly and suited for a format that allows reuse of the materials. Anion exchange material uses a process that requires elution with high salt concentrations that can inhibit nucleic acid assays and requires further processing steps to remove. Cellulose has been used as a cost-effective matrix to bind DNA, but compared to other solid phase methods, cellulose has less reproducible results.

Silica membrane provides a cost-effective, reproducible, and simple process for single use DNA extraction in a point-of-use setting. The use of silica as a substrate for DNA extraction in a microfluidic device has been demonstrated in many different fashions. Silica membrane is used by exposing the lysed cells in a mixture with a chaotropic agent to the silica membrane. While DNA and silica are in the presence of a chaotropic agent such as guanidinium thiocyanate (GuSCN), the DNA will bind to the silica. Any remaining proteins or other unwanted molecules can be washed through with solutions containing the chaotropic agent. After the membrane has been washed of unwanted molecules, the DNA can be eluted by washing a solution that does not contain the chaotropic agent. The silica membrane-based method of DNA extraction is a simple, rapid method that can be applied to a variety of suitable microfluidic devices.

Nucleic Acid Amplification Tests (NAATs) allow for the highly sensitive detection of specific DNA and RNA segments for a variety of applications including pathogen detection and diagnosis of genetic diseases. A primary advantage of NAATs is that in many cases only a small amount of genetic material is needed because the genetic material is typically amplified many times over, decreasing the need for extremely sensitive detection devices. For detecting mycotoxin-producing fungi, NAATs allow for the specific detection of a small amount of fungus using a less sensitive, portable instrument. The first NAAT invented, Polymerase Chain Reaction (PCR) has long been considered the gold standard of NAATs. The use of thermostable polymerase to synthesize genetic material allowed for small amounts of specific genetic code to be amplified to easily detectable amounts rapidly. As new methods for nucleic acid amplification have been developed, their feasibility as point-of-use tests has increased. Isothermal NAATs provide the advantage of smaller heating devices compared to PCR, which requires a large thermocycler with precise temperature control. Most reagents used in NAATs require storage in the freezer while in a liquid state, but paper based NAATs have negated the requirement for freezer storage of reagents, increasing portability.

Loop mediated isothermal amplification (LAMP) is currently a widely used isothermal amplification technique. LAMP utilizes at least four primers to rapidly amplify a highly specific region of DNA. During the forward LAMP amplification process, stem-loop DNA structures are formed through synthesis of the complimentary DNA target strand. The stem-loop DNA structures can also be used as templates for the backward primers, generating more DNA structures. Because of the use of forward and backward primers and the use of product DNA strands as future cycle targets, each cycle increases the target sequence 6-fold. A total accumulation of 10⁹ copies can be created in an hour using LAMP with four primers.

The LAMP reaction can be further accelerated by adding two “loop primers” in addition to the necessary four primers for a total of six primers. The addition of the “loop primers” to the assay can create a detectable amount of DNA in less than 30 minutes. As shown in FIG. 1.2, “dumbbell” structures are formed by replication using the forwards (F3 and FIP) and backwards (B3 and BIP) primers. The F2 (or B2) section of the FIP (or BIP) primer will first hybridize to the F2c (or B2c) complimentary sequence on the target DNA. The F3 (or B3) primer will hybridize to the F3c (or B3c) region of the target DNA, ensuring that the F1c (or B1c) region of the FIP (or BIP) primer is displaced from the target DNA strand. Polymerase will begin to use the dNTPs available in the environment to create the complimentary DNA sequence moving towards the opposing set of primers. Once the polymerase connects the FIP and BIP primers, the complimentary sequences F1 and F1c as well as B1 and B1c will fold back to hybridize, forming a loop structure. The resultant structure can be used as a target strand for future reactions, creating complex loop-stem structures as polymerase continues to create new complimentary sequences. The addition of the loop primers allows even more complex structure to be generated through the reaction.

LAMP has other advantages over PCR for point-of-use settings in addition to being an isothermal technique and being able to perform in a paper matrix. Other matrices with appropriate chemical surface modifications may also be used in some examples of the disclosed technology as shown in Table 1.

TABLE 1
Alternative matrix materials for LAMP.

| Membrane | Surface Charge | Fluorescence |
|---|---|---|
| Regenerated | Negative | High |
| cellulose | | |
| Nitrocellulose | Negative | High |
| Cellulose | Negative | High |
| Cellulose nitrate | Slightly negative | High |
| Nylon | Negative | High |
| Copy paper | Neutral (hydrophobic) | Low |
| Polypropylene | Neutral (hydrophobic) | Low |

Another advantage of LAMP is the ability to easily couple the reaction with fluorescence for real time detection. Fluorescence detection is a method that can be portable, inexpensive, and sensitive. Compared to PCR, LAMP is also more tolerant of matrix inhibitors. Inhibitory chemicals can exist in biological matrices that affect NAATs. Being more tolerant to matrix inhibitors reduces the burden of ensuring purity in the sample, which is difficult to accomplish in a field setting. LAMP reagents in liquid form are stable if kept at −20° C., but freezer storage is not suitable for in-field detection. By drying the LAMP reagents onto a membrane, the reagent stability at room temperature can be extended. In one example, suitable efficiency of LAMP reactions using LAMP reagents dried on paper after 35 days. In other examples freeze drying or lyophilizing reagents can extend shelf life at room temperature 28 days or even 3 months. Increased shelf life is desirable for applications of in-field use of NAATs.

A variety of detection methods exist for detecting the success of a LAMP reaction. Each time an amplicon is generated during a LAMP reaction, a pyrophosphate molecule is generated. The formation of magnesium pyrophosphates in solution will increase the turbidity of the solution, which can be measured. Colorimetric assays will change the color of dyes depending upon the amplification of DNA but rely on the user to determine the color change. Fluorescence assays will unquench a fluorescent probe to make the DNA amplification detectable. Fluorescence assays allow for the use of simple, inexpensive and portable detection devices because fluorescence does not require high sensitivity to detect.

Microfluidics is a rapidly growing area of research, especially in biologic diagnostic tests. Advantages of microfluidics include the ability to take advantage of certain fluid dynamics principles, lower reagent volumes and costs, low sample volume, high sensitivity to low concentrations, and the ability to convert a procedure from a complex laboratory method to a simple in-field or point-of-care (POC) method. Because of the wide range of applications for microfluidics, various fabrication methods for fabrication of microfluidic devices have been developed.

Numerous microfluidic devices have been developed for the application of DNA detection in a clinical setting. Many of these devices use paper as a membrane for drying reagents for improved shelf life. While these devices are suitable for a clinical setting, many do not meet the portability or ease-of-use requirements (e.g., no pipetting) that are desirable for in-field use. Therefore, a new design for in-field detection was developed.

Soft lithography is a common technique for fabricating microfluidic devices. Soft lithography uses an elastomeric material such as polydimethylsiloxane (PDMS) to mold or print microchannels. Other common techniques include using paper as fluidic channels, machining glass or polymers, and digital microfluidics that takes advantage of electrowetting to transfer small amounts of fluid. Machining or laser engraving poly(methyl methacrylate) (PMMA) has become a popular microfluidic fabrication technique because of the rapidity of the process, low cost of PMMA, and ability to fabricate without a clean room. The engraved PMMA can be covered with a layer of tape containing an unreactive adhesive to create the microfluidic channels in a quick and inexpensive method. Fabricating microfluidic devices through laser ablation of PMMA and application of tape is an inexpensive and quick method that typically does not require any specialized equipment other than a laser engraver. Other methods of forming channels and chambers in a microfluidic device according to the disclosed technology may also be used. For example, vector ablation, chemical etching, waterjet, or traditional machining of material may also be used.

The most widely used methods of fluid distribution in microfluidics are pressure-driven and electrokinetic-driven fluids. In paper fluidics and digital fluidics, electrokinetic forces are used by changing the hydrophobicity of the environment surrounding the fluid. Microfluidic channels created from polymers or glass can use either pressure-driven or electrokinetic-driven flow. Inside channels, electrokinetic forces are often referred to as capillary forces. Electrokinetic forces are generated through electric fields created by the environment of the microfluidic channel such as negatively charged silanol groups on the surface of a glass microfluidic device. In the case of acrylic-tape hybrid microfluidics, the tape can be configured for pressure-driven or electrokinetic-driven flow. A hydrophilic adhesive on the tape will generate flow through interactions with aqueous solutions, while hydrophobic adhesive will not attract aqueous solutions, requiring fluid to be driven by pressure.

Fluid distribution using hydrophobic tape is pressure-driven and based on the resistance generated by the channels. The change in pressure (P) is proportional to both the resistance (Rfluid) of the channel and the flow rate (Q) of the liquid as shown in equation:

$$Δ P = {R}_{fluid} Q$$

Channel resistance is a function of width (w), height (h), shape, length (L), and fluid viscosity (μ). Larger channel height and width will lead to decreased fluidic resistance, but longer channel length will increase fluidic resistance. While shape does play a role in fluidic resistance, most geometries fall within the range shown in equation:

$$\frac{12 μL}{{wh}^{3}} < {R}_{fluid} < \frac{32 μL}{{wh}^{3}}$$

The change in pressure is synonymous with the pressure required for the fluid to reach that area of the microfluidic channel. Fluid flow can be controlled by altering the above variables to change the pressure required for fluid flow to a particular area of a device.

LAMP is temperature dependent because at higher temperatures DNA polymerase will denature, and at lower temperatures DNA polymerase is not as efficient. Therefore, an optimal temperature range must be evaluated. To find the optimal temperature range, in-tube LAMP experiments were conducted at 60, 63, 64, 65, 66, and 70° C. The time was held constant at one hour for each reaction. The solution from each reaction was placed in a gel electrophoresis cassette with a DNA ladder. Gel electrophoresis was run on the solutions using a gel electrophoresis device, and the results were analyzed visually to determine the reaction temperature that had the highest yield. It was determined that 65° C. led to the highest yield of DNA, but 63° C.-70° C. yielded suitable results.

After the optimal temperature was determined, different time lengths of the reaction at 65° C. were tested. Typical time lengths range from 30 minutes to one hour for LAMP reactions. For the application of in-field detection, a shorter reaction time is desirable. For this experiment, the times ranged from 35 to 70 minutes by 5-minute increments. The results showed that the LAMP reaction produces a detectable number of products at 35 minutes, but that the total amount of products appears to increase as the reaction continues to 70 minutes or longer. For further testing, 50 minutes was determined to be an acceptable length of time to be confident that the LAMP reaction had amplified the target DNA sufficiently. Longer or shorter times may also be used.

For the assay to be useful in detecting Fusarium graminearum, the primers must be specific. To test the specificity of the primers, various Fusarium species were tested with the LAMP assay. DNA from cultured of Fusarium graminearum, Fusarium cerealis, Fusarium subglutinans, Fusarium avenaceum, Fusarium acuminatum, Fusarium nygamai, Fusarium tricinctum were obtained for testing. The DNA samples were diluted to 10 ng/mL and tested at the optimal conditions previously determined to be 65° C. for 50 minutes. The results demonstrated that the LAMP assay had some specificity, but it amplified DNA from both Fusarium cerealis and Fusarium nygamai. Testing positive for two other fungal species was determined to be acceptable for this example study because the microfluidic device has the potential to multiplex multiple LAMP assays with different primers that could further select for specific species.

The microfluidic device in one example was manufactured by engraving a pattern into a polymethyl methacrylate (PMMA) structure 50 that was 6″×6″× 5/64″. The fluidic channel 56 designs were created and engraved and inlet and outlet holes were cut into the PMMA using a laser 54. The paper or plastic matrices pre-soaked in the LAMP reagents 58 were placed inside of the appropriate channels (a LAMP reaction chamber) 52. A silica membrane was cut into 8 layers of 4 mm by 5 mm squares and placed in the appropriate location. Afterwards, Microfluidic Diagnostic Tape 60 was placed over the channels.

A laser printer engraved the pattern of distribution channels and LAMP reaction chambers over one or more passes depending on the size and power of the laser printer. In one example the pattern was engraved at a 45° angle to ensure that horizontal and vertical channels were created identically as the laser head moved diagonally across the PMMA. The approximate depth of LAMP chambers was measured to be 1.0 mm, although larger or smaller chambers may be used. The inlet, outlet, silica membrane chamber, and outer border were all created by cutting through the PMMA with a laser. After cutting the silica membrane chamber, a small piece of microfluidic tape was used to cover the other side of the hole. A female-to-female luer-lock was placed over the inlet hole, and heated ethylene-vinyl acetate (EVA) was extruded around the base of the luer-lock to form a water-tight seal when cooled and hardened.

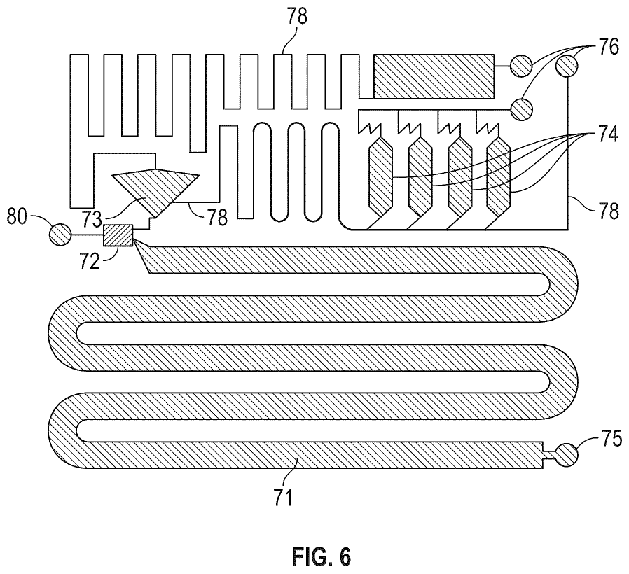

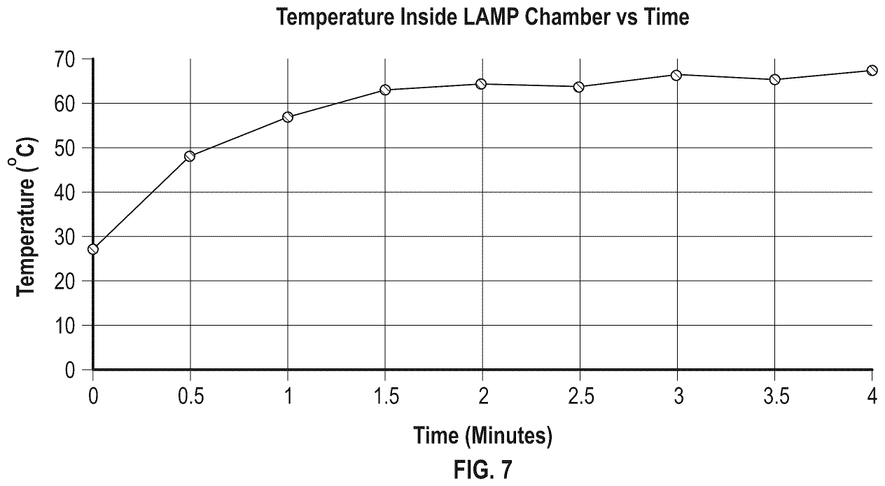

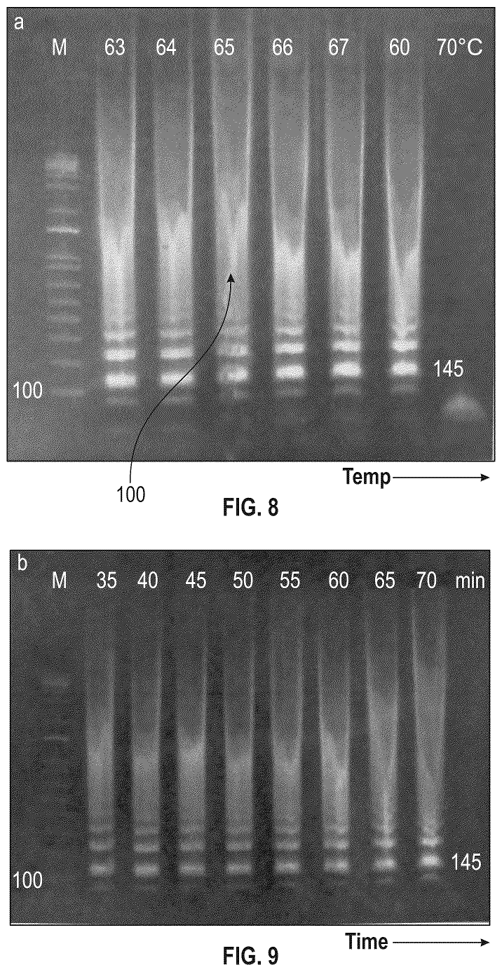

The microfluidic channel design in one example works by directing fluid flow based on the resistance of the channels and the covering and uncovering of the air outlet holes 75, 76. The outlet holes 75, 76 can simply be closed and opened by placing microfluidic tape over them. Fluidic resistance is increased by decreasing the cross-sectional area of the fluidic channels 78 and increasing the length of the channels. By using these two methods, fluid can be directed through the silica membrane 72 from the inlet 80 into either the waste area 70, 71, 73 or the LAMP reaction chambers 74. Each LAMP reaction chamber holds approximately 25 μL of fluid in one example. The engraved pattern is shown in FIG. 6.

In one example, the upper outlets 76 were covered by microfluidic tape, so the upper channels required a large amount of pressure of the liquid to enter because the air inside must be compressed for any liquid to travel. The liquid travelled through the silica membrane into Waste Area 71 while the upper outlets 76 were covered by the microfluidic tape. DNA and chaotropic agent were pushed through silica membrane and the remaining liquid flowed into Waste Area 71. Then, the wash reagents containing chelating agents can be pushed through the silica membrane into Waste Area 71 to remove any proteins or other unwanted molecules from the membrane. The silica membrane then contained DNA that can be moved to the LAMP reaction chambers.

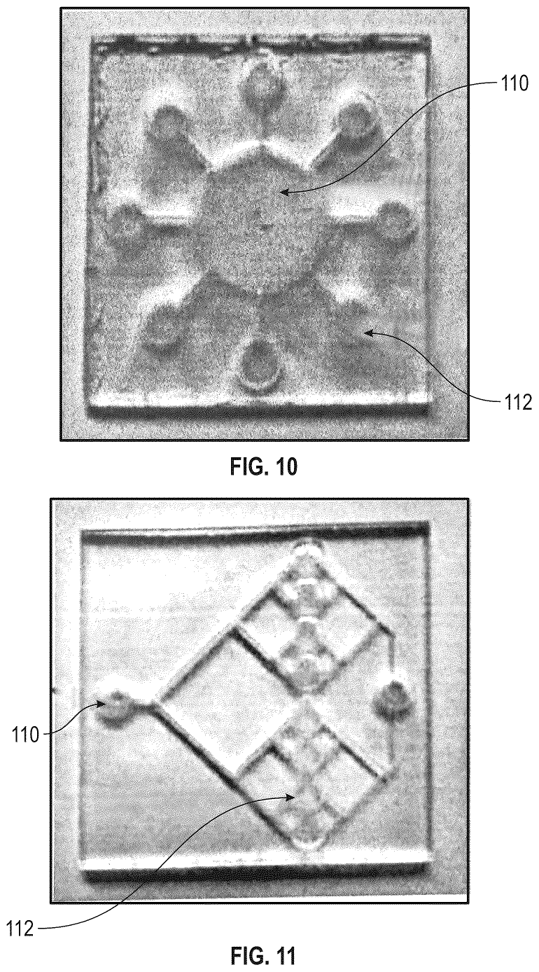

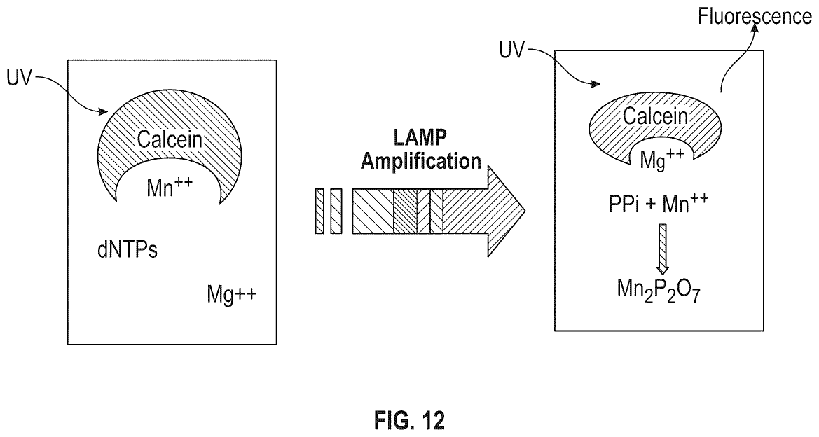

Continuing with the present example, after the DNA was bound to the silica and washed, the microfluidic tape was removed from the upper outlets 76 and tape was placed over the lower outlet 75. Now, lower Waste Area 71 required much more pressure for the liquid to enter because the remaining air inside must be compressed for the liquid to move. The liquid travelled first into Waste Area 73, which is filled with liquid before the liquid continues to travel to Waste Area 70 and the LAMP reaction chambers. Waste Area 73 is filled before the liquid travels through the channels due to the high fluidic resistance of the channels generated by their small cross-sectional area and long length. Once Waste Area 73 was filled, the remaining chaotropic agent in the silica membrane area was evacuated. The DNA was then unbound from the silica and travelled to both the Waste Area 70 and the LAMP reaction chambers 74. In this example, fluid was distributed by adding and removing tape and through the inherent resistance of the microfluidic channels. The design shown in FIG. 6 is one example of possible configurations. Other examples are shown in FIG. 10-11 which use differing input port 110 locations as well as different read port 112 locations. Other suitable configurations may also be used.

In one example a heating device was created to heat the device to 65° C. The device consisted of an aluminum block that the microfluidic device was placed on, a thermistor for reading the temperature of the block, a heating element, and control device for controlling the heating element based on the temperature of the block. To validate the heating time and temperature stability, a k-type wire thermocouple may be placed inside of the LAMP reaction chamber of a microfluidic device filled with water. In one example the LAMP reaction will occur at 65±2° C. In other examples, higher or lower temperatures may be used.

In one example, a contaminated wheat sample was added to the tubes containing the beads. Next, 750 μL of buffer containing trometamol (Tris) buffer and ethylenediaminetetraacetic acid (EDTA) as a chelating agent for nucleases is also added to the tube. A bead beater, a vortexer or other suitable device may be used for a suitable period of time (e.g., 5 minutes) to sufficiently pulverize the grain samples.

For the microfluidic DNA extraction procedure, the next step was to 1 ml syringe to aspirate the liquid from the tube. The liquid was then mixed with 1,200 μL of Genomic Lysis Buffer containing guanidinium thiocyanate as a chaotropic agent and glycerol for ensuring the DNA spreads across the membrane. Tape was placed over the upper outlets of a microfluidic chip containing no LAMP reagents to force the liquid to travel through the silica membrane and into Waste Area 75. A 3 mL syringe was connected to the luer-lock and used to inject the solution into the microfluidic chip. Next, 200 μL of DNA Pre-Wash Buffer containing guanidinium chloride as a chaotropic agent and isopropyl alcohol to help remove unwanted proteins and other molecules was injected with a 3 mL syringe. After, 500 μL of g-DNA Wash Buffer containing ethanol and isopropyl alcohol for washing out unwanted molecules was injected with a 3 mL syringe. Finally, DNA Elution containing 2-amino-2-(hydroxymethyl) propane-1,3-diolhydrochloride (Tris HCl) to increase pH to cause the DNA to elute and EDTA to reduce nuclease activity was injected with a 3 mL syringe. After waiting 5 minutes to ensure the DNA is uniformly diffused, 100 μL of fluid was aspirated from the outlet closest to the LAMP reaction chambers using a micropipette.

Optionally, to determine the DNA quantity extracted, qPCR was performed on each of the DNA extraction experiments. The primers used in the qPCR reaction target the Tri5 gene that regulates the production of trichothecene mycotoxins and are shown in Table 2-4. The primers were purchased in 100 μM stocks and diluted to 20 μM with water. The reaction mixture contained 1 μL each of both primers, 5 μL of the extracted DNA, 3 μL of nuclease-free water, and 10 μL of Chai Green qPCR Mastermix. A qPCR device was used to perform the qPCR reaction and measure the fluorescence.

The output of qPCR is Ct values, which are the number of cycles reached before the reaction reaches the fluorescence threshold. Previously, a qPCR experiment was performed using known concentrations.

The application of the microfluidic device requires that the LAMP assay detect a level of Fusarium graminearum that produces dangerous levels of DON. The amount of Fusarium graminearum DNA required to produce dangerous levels of DON can vary greatly. The lowest reported Fusarium graminearum DNA amount required to produce dangerous levels of DON vary with a lowest reported value of 25 μg of DNA per 1 kg of wheat. With 100 g of dry wheat used in each DNA extraction, the device must be able to detect at least 2.5 μg of DNA in each sample. Using the elution buffer volume, a concentration of 6.25 μg/mL must be detectable. Because LAMP has high sensitivity to small amounts of DNA, much lower concentrations were tested.

Limit of detection (LOD) experiments were performed to determine the minimum concentration of DNA required for detection in the microfluidic device assay format. To remove the variability of the DNA extraction process, a different design with only LAMP chambers was utilized with known concentrations of DNA being added to each chamber. The design shown in FIG. 6 was engraved into 3/16″ thick PMMA using a laser. The laser created chambers that had only 1 mm of PMMA thickness at the bottom of the chamber in this example.

Polypropylene membrane filters were cut to fit the LAMP chambers and pretreated with 10 μL of 10% Tween-20 solution. Then, reagents 10× ThermoPol Buffer, MgSO₄, dNTP Mix, FIP/BIP Primers, F3/B3 Primers, LoopF/LoopB Primers, Calcein, and Nuclease-free Water were dried to the membrane inside of a flow hood in seven of the chambers. The negative amplification control included all the above reagents except for the DNA and primers. The positive and negative fluorescence controls were 25 μL of 2.5 μM calcein and 25 μL of 2.5 μM calcein and manganese complex, respectively. In the seven LAMP amplification chambers, a dilution series of Fusarium graminearum DNA from culture was added starting with 10 μg/mL that was diluted 1:10 in series to 10 μg/mL. In each of the seven amplification chambers, 25 μL of DNA diluted in water was added. In the three control chambers, 25 μL of water was added. Microfluidic tape was used to cover the chambers, and the device was heated on the prototype heating device to 65° C. for 50 minutes. Then, the microfluidic device was placed inside an empty well plate with the LAMP chambers aligned with the positions of a 96 well plate. The empty well plate was placed inside of a multi-mode microplate reader with the fluorescence settings of 1 mm read height, 485 nm excitation, 528 nm emission, gain 35, and normal read speed. Each chamber aligns with two measurements in the plate reader, so results are reported as averages of the two measurements.

After the DNA extraction and LOD experiments were completed, the complete process was tested. The complete process included both the DNA extraction and the LAMP assay inside of the microfluidic chip. The polypropylene filters were laser cut to the shape of the LAMP chambers and pretreated with 10 μL of 10% Tween-20 solution. On one of the matrices, the above reagents except for the DNA are added to create the test matrix. The negative amplification control included all the above reagents except for the DNA and primers. The positive and negative fluorescence controls were 25 μL of 2.5 μM calcein and 25 μL of 2.5 μM calcein and manganese complex, respectively.

After the reagents are dried, they are placed inside the LAMP chambers of the microfluidic device. The silica membrane is placed inside of the microfluidic device, and the microfluidic tape is used to seal it. The DNA extraction procedure is followed as described in the microfluidic procedure above.

After the DNA extraction was performed, the upper outlets of the microfluidic device were covered with tape. The microfluidic device was placed on a digital dry bath preheated to 65° C. The device was heated for 50 minutes. Then, the microfluidic device was placed inside an empty well plate with the LAMP chambers aligned with the positions of a 96 well plate. The empty well plate was placed inside of a multi-mode microplate reader with the fluorescence settings of 1 mm read height, 485 nm excitation, 528 nm emission, gain 35, and normal read speed.

The previous examples discussed various methods and devices for detecting the presence of mycotoxin-producing fungi in grains or other crops. It is understood that the disclosed methods and devices may be used to detect the presence of more than one species or types of mycotoxin-producing fungus. It is also understood that the disclosed methods and devices may be adapted to detect two or more species of mycotoxin-producing fungi simultaneously and/or sequentially.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments may also be implemented in combination in a single embodiment. Conversely, various features that are described in the context of a single embodiment may also be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to nigh-infinite subcombinations or variations of a subcombination.

While the claimed technology has been illustrated and described in detail in the drawings and foregoing description, the same is to be considered as illustrative and not restrictive in character. It is understood that the embodiments have been shown and described in the foregoing specification in satisfaction of the best mode and enablement requirements. It is understood that one of ordinary skill in the art could readily make a nigh-infinite number of insubstantial changes and modifications to the above-described embodiments and that it would be impractical to attempt to describe all such embodiment variations in the present specification. Accordingly, it is understood that all changes and modifications that come within the spirit of the claimed technology are desired to be protected.

What is claimed is:

1. A method of detecting the presence of mycotoxin-producing fungus in grain in the field, comprising:
a) procuring a grain sample;
b) extracting DNA from the grain sample;
c) isothermally amplifying the extracted DNA;
d) identifying any DNA from mycotoxin producing fungus as amplified target DNA; and
e) fluorescing products of amplified target DNA reactions to yield quantitative information relating to the presence of DNA from mycotoxin-producing fungus;
wherein steps c-e are all performed within the same microfluidic chamber platform.

2. The method of detecting the presence of mycotoxin-producing fungus of claim 1, wherein the extracting comprises milling the grain sample and lysing the milled grain sample.

3. The method of detecting the presence of mycotoxin-producing fungus of claim 1, wherein the extracting comprises solid phase extraction using one of silica membranes, glass particles, diatomaceous earth, magnetic beads, anion exchange material, and cellulose matrix.

4. The method of detecting the presence of mycotoxin-producing fungus of claim 1, between step b) and step c) the sample is washed with a chaotropic agent.

5. The method of detecting the presence of mycotoxin-producing fungus of claim 1, wherein the amplifying and identifying includes a paper-based nucleic acid amplification test.

6. The method of detecting the presence of mycotoxin-producing fungus of claim 1, wherein the amplifying is a loop-mediated isothermal amplification.

7. The method of detecting the presence of mycotoxin-producing fungus of claim 6, wherein the loop-mediated isothermal amplification uses six primers.

8. The method of detecting the presence of mycotoxin-producing fungus of claim 1, wherein DNA from two or more different mycotoxin-producing fungi species are extracted, amplified, identified, and fluoresced.

9. A method of in-field detection of mycotoxin producing fungus, comprising:
a) collecting potentially infected plant tissue samples;
b) grinding potentially infected plant tissues to yield ground tissue material;
c) lysing ground tissue material to extract DNA;
d) purifying extracted DNA to yield purified DNA;
e) performing a fluorescence-based loop-mediated isothermal amplification assay on the purified DNA;
f) measuring the amount of fluorescence from DNA from mycotoxin-producing fungus to yield a quantitative measurement;
wherein steps b-c are performed in a first device and steps d-f are performed in a second device.

10. The method of in-field detection of mycotoxin producing fungus of claim 9, wherein the first device comprises a portable bead mill.

11. The method of in-field detection of mycotoxin producing fungus of claim 9, wherein the second device comprises a portable microfluidic device which includes:
a housing;
a microfluidic chamber for purifying extracted DNA and performing a fluorescence-based loop-mediated isothermal amplification assay on the purified DNA positioned within the housing and operationally connected to the bead-bashing chamber; and
a power source positioned within the housing and operationally connected to the microfluidic chamber.

12. A portable microfluidic device for extracting fungal DNA from grain samples and performing qualitative and quantitative analysis of the same, comprising:
a housing;
a bead-bashing chamber enclosed within the housing for extracting DNA from plant tissue samples;
a microfluidic chamber for purifying extracted DNA and performing a fluorescence-based loop-mediated isothermal amplification assay on the purified DNA positioned within the housing and operationally connected to the bead-bashing chamber; and
a power source positioned within the housing and operationally connected to the microfluidic chamber.

13. The device of claim 12, wherein the power source is operationally connected to the bead-bashing chamber.

14. The device of claim 12, wherein the microfluidic chamber further includes an input port, at least two fluid distribution channels fluidically connected to the input port, at least one outlet port fluidically connected to each of the at least two fluid distribution channels,
wherein fluid flow through the at least two fluid distribution channels may be controlled by selectively covering the outlet ports.

15. The device of claim 14, wherein the outlet ports are selectively covered by hydrophobic microfluidic tape.

16. The device of claim 14, further comprising at least one LAMP reaction chamber fluidically connected to one of the fluid distribution channels.

17. The device of claim 12, further comprising a heating device configured and arranged to warm the microfluidic chamber to at least 65 degrees C.

18. The device of claim 12, wherein the microfluidic chamber is made from PMMA.

19. The device of claim 12, wherein the input port is a luer lock style port.
